(19) United States
(12) Patent Application Publication
(10) Pub. No.: US 2022/0192528 A1
(43) Pub. Date: Jun. 23, 2022

(54) HEART RATE SENSOR AND ELECTRONIC DEVICE FOR COLLECTING HEART RATE

(71) Applicant: Weifang Goertek Microelectronics Co., Ltd., Weifang, Shandong (CN)

(72) Inventors: WENTAO WANG, Weifang, Shandong (CN); Huabin Fang, Weifang, Shandong (CN); Dexin Wang, Weifang, Shandong (CN)

(73) Assignee: Weifang Goertek Microelectronics Co., Ltd., Weifang, Shandong (CN)

(21) Appl. No.: 17/606,485

(22) PCT Filed: Dec. 6, 2019
(86) PCT No.: PCT/CN2019/123554

§ 371 Date: Oct. 26, 2021

(30) Foreign Application Priority Data
Apr. 26, 2019 (CN) 201910343047.0

(51) Int. Cl. A61B 5/024; A61B 5/1455

(52) U.S. Cl. CPC A61B 5/02427; A61B 2560/0204; A61B 5/14551

(57) ABSTRACT

Disclosed are a heart rate sensor and an electronic device for collecting heart rate. The heart rate sensor includes a substrate provided thereon with modules optically isolated from each other, the modules including: a first light wave emitting module, configured to emit a green light wave for testing heart rate; second light wave emitting modules, configured to emit a red light wave and an infrared light wave for testing blood oxygen and the heart rate; a first light wave receiving module and a second light wave receiving module, configured to receive a reflected green light wave, a reflected red light wave and a reflected infrared light wave; wherein, the first light wave receiving module and the second light wave receiving module are located on two respective sides of the first light wave emitting module, and the second light wave emitting modules are provided in two groups.

### CROSS-REFERENCE TO RELATED APPLICATION

This application is a National Stage of International Application No. PCT/CN2019/123554, filed on Dec. 6, 2019, which claims priority to Chinese Patent Application No. 201910343047.0, filed on Apr. 26, 2019, both of which are hereby incorporated by reference in its entirety.

### TECHNICAL FIELD

The present disclosure relates to the field of optical sensor, and more specifically to a heart rate sensor and an electronic device for collecting heart rate.

### BACKGROUND

In modern society, due to people's unreasonable lifestyle and dietary pattern, some cardiovascular and cerebrovascular such as hypertension and coronary heart disease have gradually become common and frequently-occurring diseases in clinical medicine, and most of these diseases are chronic diseases and can only be controlled but cannot be cured, which requires a patient to go to a hospital for regular checkups and take medications. However, this still cannot avoid occurrence of emergencies, so it is necessary to monitor the patient's heart rate changes in real time, so as to find a problem in time. With an improvement of the people's living standards, more and more people begin scientific exercise to maintain their health, and in order to ensure rationality of the exercise and its effect, using the heart rate device to monitor heart rate to make reasonable exercise plans has become more and more widely recognized and accepted by people.

In recent years, blood oxygen sensors, blood pressure sensors, and heart rate sensors have been used in products such as smart devices, portable measurement devices, and portable medical instruments. Currently, the heart rate sensors on the market are generally divided into two forms including a separate scheme and an integrated scheme. Due to the use of discrete components, the separation scheme needs to be divided into multiple times during assembly, which produces a large assembly position error, increases a positional inconsistency between a light-emitting element and a receiving element, and results in poor consistency of received signals. Moreover, the separation scheme mostly requires grating design in the matching structure of a whole machine, which leads to disadvantages such as a large size, and in the continuous platform update, the separation scheme needs to continuously select different LED and PD accessories to match, which is more cumbersome; the integration scheme in the prior art mostly adopts the integration method of photodiode (PD) and LED lamp, and therefore has disadvantages such as a large size and low integration.

In view of the above, it is necessary to provide a new technical solution to solve the above problems.

### SUMMARY

An object of the present disclosure is to provide a new technical solution of a heart rate sensor and an electronic device for collecting heart rate.

According to a first aspect of the present disclosure, a heart rate sensor is provided, including a substrate provided thereon with modules optically isolated from each other, the modules including:

a first light wave emitting module, the first light wave emitting module is configured to emit a green light wave for testing heart rate;

second light wave emitting modules, the second light wave emitting modules are configured to emit a red light wave and an infrared wave for testing blood oxygen and the heart rate;

a first light wave receiving module and a second light wave receiving module, the first light wave receiving module and the second light wave receiving module are configured to receive a reflected green light wave, a reflected red light wave and a reflected infrared light wave;

the first light wave receiving module and the second light wave receiving module are located on two respective sides of the first light wave emitting module, and the second light wave emitting modules are provided in two groups, with one group located on one side of the light wave receiving module away from the first light wave emitting module and the other group located on one side of the second light wave receiving module away from the first light wave emitting module.

Optionally, further including an isolation grating wall covering the substrate, the isolation grating wall is provided with a plurality of accommodating grooves in communication with the substrate, and the accommodating grooves have positions respectively corresponding to those of the first light wave emitting module, the second light wave emitting modules, the first light wave receiving module and the second light wave receiving module.

Optionally, the substrate and the isolation grating wall are integrally formed, or the substrate and the isolation grating wall are bonded or welded together.

Optionally, further including an analog front end module and a power management module provided on the substrate, one of the first light wave receiving module and the second light wave receiving module is mounted on the analog front end module, and the other is mounted on the power management module.

Optionally, the analog front end module and/or the power management module are embedded in the substrate.

Optionally, the first light wave emitting module includes three green LED chips which are distributed in a straight line.

Optionally, each group of the second light wave emitting module includes one red LED chip and one infrared LED chip which are distributed in a straight line parallel to the three green LED chips.

Optionally, the accommodating grooves of the isolation grating wall are filled with transparent colloid therein, or provided with transparent glass thereon.

Optionally, the first light wave emitting module and the first light wave receiving module and/or the first light wave emitting module and the second light wave receiving module have a distance of 2.3-3.2 mm therebetween; the second light wave emitting module and the second light wave receiving module have a distance of 6-10 min therebetween.

According to a second aspect of the present disclosure, an electronic device for collecting heart rate is provided, including the heart rate sensor described above.

By providing a second light wave emitting module on both sides of the substrate for emitting a red light waves and an infrared light wave for testing blood oxygen and heart rate, the heart rate sensor of the disclosure can effectively increase the detection range and signal intensity of the blood oxygen signal, and can effectively improve the monitoring accuracy of the heart rate sensor. Even if the heart rate sensor is tilted and shifted, more accurate data can be measured.

Other features and advantages of the present disclosure will become apparent from the following, detailed description of exemplary embodiments of the present disclosure with reference to the accompanying drawings.

### BRIEF DESCRIPTION OF THE DRAWINGS

The accompanying drawings, which are incorporated in and constitute a part of the description, illustrate embodiments of the present disclosure and, together with the description thereof, serve to explain the principles of the present disclosure.

FIG. 1 is a schematic diagram of the structure of a heart rate sensor of the present disclosure.

### DETAILED DESCRIPTION

Various exemplary embodiments of the present disclosure will now be described in detail with reference to the accompanying drawings. It should be noted that the relative arrangement, numerical expressions and numerical values of the components and steps set forth in these examples do not limit the scope of the disclosure unless otherwise specified.

The following description of at least one exemplary embodiment is in fact merely illustrative and is in no way intended as a limitation to the present disclosure and its application or use.

Techniques, methods, and apparatus known to those of ordinary skill in the relevant art may not be discussed in detail but where appropriate, the techniques, methods, and apparatus should be considered as part of the description.

Among all the examples shown and discussed herein, any specific value should be construed as merely illustrative and not as a limitation. Thus, other examples of exemplary embodiments may have different values.

It should be noted that similar reference numerals and letters denote similar items in the accompanying drawings, and therefore, once an item is defined in a drawing, and there is no need for further discussion in the subsequent accompanying drawings.

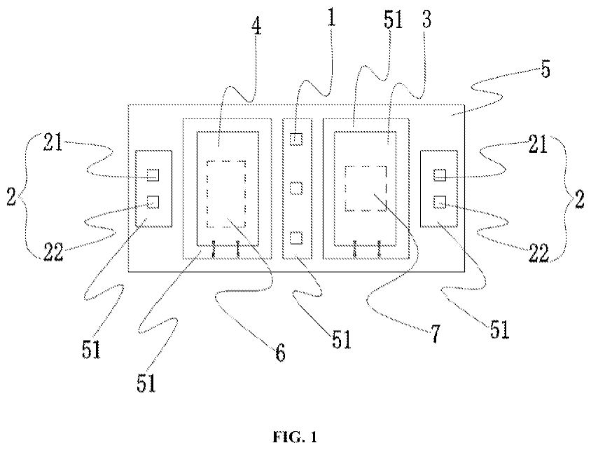

Referring to FIG. 1, a heart rate sensor is provided by an embodiment of the present disclosure, and includes a substrate which is a PCB or BT board, wherein a circuit layout for the heart rate sensor is preset in the PCB or BT board. The substrate is provided with a first light wave emitting module 1 configured to emit green light waves required for testing heart rate and second light wave emitting modules 2 configured to emit red light waves and infrared light waves required for testing blood oxygen and heart rate. The substrate is also provided with a first light wave receiving module 3 and a second light wave receiving module 4 which may use photodiodes (PD) for converting optical signals into electrical signals and are configured to receive the reflected green light waves, red light waves and infrared light waves. By providing two light wave receiving modules, the present disclosure can effectively increase detection ranges for receiving the reflected light, respectively receive the red light waves, infrared light waves and green light waves reflected from skin or blood, and respectively test blood oxygen saturation data and heart rate data, etc. so as to improve detection effect. The first light wave receiving module 3 and the second light wave receiving module 4 are located on two respective sides of the first light wave emitting module 1. The second light wave emitting modules 2 are set in two groups with one group located on one side of the first light wave receiving module 3 away from the first light wave emitting module 1 and the other group located on one side of the second light wave receiving module 4 away from the first light wave emitting module 1. That is, the first light wave receiving module 3 is located between the first light wave emitting module 1 and a group of the second light wave emitting module 2, and the second light wave receiving module 4 is located between the first light wave emitting module 1 and the other group of second tight wave emitting modules 2. By providing a group of the second light wave emitting module 2 on both sides of the substrate, the embodiment of the present disclosure can increase detection range and signal strength a blood oxygen signal, and can effectively improve accuracy of heart rate sensor monitoring, if only one group of the second light wave emitting modules 2 is provided, then when the heart rate sensor is tilted and shifted, its monitoring effect will be affected, white providing two groups of the second light wave emitting modules 2 can effectively avoid this problem, and even if the heart rate sensor is tilted and shifted, it can measure more accurate data.

Further, the heart rate sensor further includes an isolation grating wall 5 covering the substrate. The isolation grating wall 5 is made of opaque material, and is used to isolate the first light wave omitting module 1, the second light wave emitting module 2, the first light wave receiving module 3, and the second light wave receiving module 4 from each other. In this way, the present disclosure can effectively prevent optical signals emitted by the first light wave emitting module 1 and the second light wave emitting module 2 from being directly sensed by the first light wave receiving module 3 and the second light wave receiving module 4, so that the first light wave receiving module 3 and the second light wave receiving module 4 will not interfere with the first light wave emitting module 1 and the second light wave emitting module 2. The isolation grating wall 5 is provided with a plurality of accommodating grooves 51 in communication with the substrate, and the positions of the accommodating grooves 51 respectively correspond to the those of the first light wave emitting module 1, the second light wave emitting modules 2, and the first light wave receiving module 3 and the second light wave receiving module 4. That is, the first light wave emitting module 1, the first light wave receiving module 3, the second light wave receiving module 4, and each group of the second light wave emitting module 2 are each located at a position of the accommodating groove 51.

In one embodiment, the heart rate sensor further includes an analog front end module (AFE) 6 and a power management module 7 provided on the substrate. One of the first light wave receiving module 3 and the second light wave receiving module 4 is mounted on the analog front end module 6, and the other of the first light wave receiving module 3 and the second light wave receiving module 4 is mounted on the power management module 7. The analog front end modules 6 is used to feedback and convert signals, and the power management module 7 is used to supply power to the first light wave emitting module 1, the second light wave emitting modules 2, the first light wave receiving module 3, and the second light wave receiving module 4.

In one embodiment, the power management module 7 is a PMIC chip which can control multiple circuits at the same time. In one embodiment, the analog front end module 6 and/or the power management module 7 are embedded in the substrate, which can reduce the thickness of the entire heart rate sensor from about 0.7-0.8 mm to about 0.4 mm, thus effectively reducing the size of the heart rate sensor.

In one embodiment, the first light wave emitting module 1 includes three green LED chips distributed in a straight line. The green light waves emitted by the green LED chips can measure a change of blood density when it flows in blood vessels, and can test the heart rate data after further calculation. The reason for providing three green LED chips is that the green light emitted in this way has a higher intensity, and more green light signals are returned from the blood. In addition, the three green LED chips arranged in the straight line can increase the detection range, and when the heart rate sensor tilts and shifts, the light signal will also be fed back, instead of no signal being received when only a green LED chip is provided.

In one embodiment, each group of the second light wave emitting module 2 includes a red LED chip 21 and an infrared LED chip 22. The red LED chip 21 and the infrared LED chip 22 can be used to test the blood oxygen saturation data and the heart rate data, etc. The red LED chip 21 and the infrared LED chip 22 in each group of the second light wave emitting module 2 are distributed in a straight line parallel to the three green LED chips, and such distribution can increase the detection range.

In one embodiment, the distance between the first light wave emitting module 1 and the first light wave receiving module 3 and/or the distance between the first light wave emitting module 1 and the second light wave receiving module 4 is 2.3-3.2 mm (due to requirements of the size of the module, it is not limited to reach that the distance between each light wave receiving module and the first light wave emitting module 1 is this distance; but in order to reduce the size of the module, the distance between at least one light wave receiving module and the light wave emitting module 1 must be 2.3-3.2 mm, and of course, it is best that the distance between each light wave receiving modules and the first light wave emitting module 1 is within this range); the distance between the second light wave emitting module 2 and the second light wave receiving module 4 is 6-10 mm. By adjusting the distance between the first light wave emitting module 1 and the first light wave receiving module 3, and the distance between the second light wave emitting module 2 and the second light wave receiving module 4, a better optical distance can be achieved, so as to make the first light wave receiving module 3 well receive the green light wave reflected from the skin or blood, make the second light wave receiving module 4 well receive the red light wave and infrared light wave reflected from the skin or blood, or make the first light wave receiving module 3 and the second light wave receiving module 4 well receive the red light wave, infrared light wave and green light wave reflected from the skin or blood at the same time, which are calculated multiple times and averaged to help to achieve accurate detection.

In one embodiment, the substrate and the isolation grating wall 5 are integrally formed. In this embodiment, when packaging the heart rate sensor in SIP, first the isolation grating wall 5 is integrated with the substrate by injection molding; then the first light wave emitting module 1, the two groups of the second light wave emitting modules 2, the analog front end module 6 and the power management module 7 are mounted on the substrate at positions corresponding to the accommodating grooves 51 of the isolation grating wall 5 by patching, and of course, the analog front end module 6 can be embedded in the substrate in advance, and then one of the first light wave receiving module 3 and the second light wave receiving module 4 is mounted on the analog front end module 6 while the other is mounted on the power management module 7; next, electrodes of the first light wave emitting module 1, two groups of the second light wave emitting modules 2, the first light wave receiving module 3 and the second light wave receiving module 4 are connected to PCB pads of the substrate by wire bonding; and finally the accommodating grooves 51 of the isolation grating wall 5 is filled with transparent colloid, or the accommodating grooves 51 of the isolation grating wall 5 are provided with transparent glass thereon by injection molding pressing or pasting.

In one embodiment, the substrate and the isolation grating wall 5 are bonded or welded together. In this embodiment, before packaging the heart rate sensor in SIP, first the isolation grating wall 5 matching the shape and size of the substrate is injection molded through a mold, and the isolation grating wall 5 has accommodating grooves 51; then the first light wave emitting module 1, the two groups of the second light wave emitting modules 2, the analog front end module 6, and the power management module 7 are mounted on the substrate by patching, and of course, the analog front end module 6 can be embedded in the substrate in advance, and it should be noted that when mounting, positions of the first light wave emitting module 1, the two groups of the second light wave emitting modules 2, the analog front end module 6, and the power management module 7 need to correspond to the positions of the accommodating grooves 51; next, one of the first light wave receiving module 3 and the second light wave receiving module 4 is mounted on the analog front end module 6 while the other is mounted on the power management module 7; then, the first light wave emitting module 1, the two groups of the second light wave emitting module 2, the first light wave receiving module 3 and the second light wave receiving module 4 are connected to PCB pads of the substrate by wire bonding; finally, the pre-injected isolation grating wall 5 is integrated with the substrate by bonding or ultrasonic welding. Accommodating grooves 51 of the isolation grating wall 5 can be injected with transparent glass in advance by two-color injection molding, and can also be filled with transparent colloid.

In the above two embodiments, whether it is transparent glass or transparent colloid, the light waves emitted by the first light wave emitting module 1 and the second light wave emitting modules 2 can be transmitted outward through the accommodating grooves 51 in the isolation grating wall 5 so as to be received by the human skin or blood, while the first light wave receiving module 3 and the second light wave receiving module 4 can respectively receive the light waves reflected from the human skin or blood and entering from the corresponding accommodating grooves 51. In addition, filling the accommodating grooves 51 of the isolation grating wall 5 with the transparent colloid can not only protect the first light wave emitting module 1, the second light wave emitting modules 2, the first light wave receiving module 3, and the second light wave receiving module 4, but also can fix the above-mentioned components after the transparent colloid is cured without affecting the function of each component. The way in which the transparent glass is provided by injection molding in the accommodating grooves 51 of the isolation grating wall 5 can also protect the first light wave emitting module 1, the second light wave emitting modules 2, the first light wave receiving module 3, and the second light wave receiving module 4, and it should be noted at this point that the thickness of the transparent glass should not affect the size of the entire heart rate sensor. It should be noted that whether it is the transparent colloid or the transparent glass, its refractive index needs to be close to that of the human skin so as to avoid affecting test effects.

In actual use, the heart rate sensor is measured in a manner close to the human skin. The light emitted by the first light wave emitting module 1 and the second light wave emitting module 2 is directed to the human skin, and a part of the light will be absorbed by skin soft tissues while the other part is reflected back from the skin or blood and received by the first light wave receiving module 3 and the second light wave receiving module 4 respectively. A difference in oxygen content in the blood will cause a difference in absorption rate of the red light and the infrared light, which will cause the reflected light to have a slight change and cause output current of the first light wave receiving module 3 and the second light wave receiving module 4 to change. This change is converted by the analog front end module 6 and sent to other components such as a processor for further processing, for example, by comparing the intensity of the red light signal with that of the infrared light signal, so as to calculate the oxygen content in the blood, that is, obtain a blood oxygen value. Moreover, when the heart beats, blood will flow in the skin, which will cause changes in the blood content in the skin, and then by calculating the relationship between changes of the red light signal or infrared light signal and time, the heart rate can be calculated. In addition, the heart rate data can also be tested by measuring the change of blood content with the change of the green light signal, and then calculating the relationship between the change of the green light signal and time.

An embodiment of the present invention also provides an electronic device for collecting heart rate, including the heart rate sensor as described above. The electronic device for collecting the heart rate may be an electronic product such as smart bracelets, smart watches, smart phones, and portable medical devices.

The internal space of smart wearable products or consumer electronic products such as smart bracelets, smart watches, smart phones, etc. are very compact, and the motherboard and other components of the whole machine take up a lot of space, so the capacity space left for the battery is very small, and this will cause the standby time of the whole machine to be reduced accordingly. The heart rate sensor of the embodiment of the disclosure packages wafers of the analog front end module, the light wave receiving module, the light wave emitting modules, the power management module and other components into

1. A heart rate sensor, comprising a substrate provided thereon with modules optically isolated from each other, the modules comprising:
a first light wave emitting module, configured to emit a green light wave for testing a heart rate;
two or more second light wave emitting modules, configured to emit a red light wave and an infrared light wave for testing a blood oxygen level and the heart rate; and
a first light wave receiving module and a second light wave receiving module, configured to receive a reflected green light wave, a reflected red light wave and a reflected infrared light wave;
wherein the first light wave receiving module and the second light wave receiving module are located on two respective sides of the first light wave emitting module, and the two or more second light wave emitting modules are provided in two groups, with a first group located on a side of the light wave receiving module away from the first light wave emitting module and a second group located on a side of the second light wave receiving module away from the first light wave emitting module.

2. The heart rate sensor according to claim 1, further comprising an isolation grating wall covering the substrate, the isolation grating wall having a plurality of accommodating grooves in communication with the substrate, wherein the accommodating grooves have positions corresponding to respective positions of the first light wave emitting module, the second light wave emitting modules, the first light wave receiving module and the second light wave receiving module.

3. The heart rate sensor according to claim 2, wherein the substrate and the isolation grating wall are selected from the group consisting of being integrally formed, bonded together and welded together.

4. The heart rate sensor according to claim 1, further comprising an analog front end module and a power management module provided on the substrate, wherein a first of the first light wave receiving module and the second light wave receiving module is mounted on the analog front end module, and a second of the first light wave receiving module is mounted on the power management module.

5. The heart rate sensor according to claim 4, wherein the analog front end module and/or the power management module are embedded in the substrate.

6. The heart rate sensor according to claim 1, wherein the first light wave emitting module includes three green LED chips which are distributed in a straight line.

7. The heart rate sensor according to claim 1, wherein each of the groups of the second light wave emitting module comprises one red LED chip and one infrared LED chip distributed in a straight line parallel to the three green LED chips.

8. The heart rate sensor according to claim 1, wherein the accommodating grooves of the isolation grating wall are filled with transparent colloid therein, or provided with transparent glass thereon.

9. The heart rate sensor according to claim 1, wherein the first light wave emitting module and the first light wave receiving module and/or the first light wave emitting module and the second light wave receiving module have a distance of 2.3-3.2 mm therebetween; and the second light wave emitting module and the second light wave receiving module have a distance of 6-10 mm therebetween.

10. An electronic device for collecting heart rate, comprising the heart rate sensor according to claim 1.
